(19) United States
(12) Patent Application Publication
(10) Pub. No.: US 2007/0282186 A1
(43) Pub. Date: Dec. 6, 2007

(54) BLOOD GLUCOSE MONITOR WITH AN INTEGRATED DATA MANAGEMENT SYSTEM

(76) Inventor: Adrian Gilmore, Pittstown, NJ (US)

Correspondence Address: TERUMO MEDICAL CORPORATION, 6200 JACKSON ROAD, ANN ARBOR, MI, 48103, US

(21) Appl. No.: 11/743,475

(22) Filed: May 2, 2007

Related U.S. Application Data
(60) Provisional application No. 60/796,685, filed on May 2, 2006.

(51) Int. Cl. A61B 5/00

(52) U.S. Cl. 600/365

(57) ABSTRACT

A method and apparatus of blood glucose monitoring is provided including the steps of acquiring results of a test of a patient's blood glucose level at a first location, converting the results of the patient's blood glucose level to glucose data, automatically transmitting the glucose data to a second location, and providing access to the glucose data from the second location for review of the glucose data. A doctor or physician can then review the blood glucose data from a remote location and provide therapy treatment from a remote location to the patient. The therapy treatment can be provided back to the patient and displayed automatically to the patient if any therapy treatment or prescription is required.

### CROSS-REFERENCE TO RELATED APPLICATION

This application claims benefit to provisional application No. 60/796,685 filed on May 2, 2006 and is incorporated herein by reference.

### FIELD OF THE INVENTION

The present invention is directed to a blood glucose monitoring (BGM) system, and more specifically, to a self blood glucose monitoring system and a passive wireless data management system that transfers the blood glucose data to a central server.

### BACKGROUND OF THE INVENTION

Glucose is a simple sugar containing six carbon atoms (a hexose), and is an important source of energy in the body and the sole source of energy for the brain. Glucose is stored in the body in the form of glycogen. In a healthy person, the concentration of glucose in the blood is maintained at around 5 mmol/l by a variety of hormones, principally insulin and glucagon. If the blood-glucose concentration falls below this level neurological and other symptoms may result, such as hypoglycemia. Conversely, if the blood-glucose level is raised above its normal level, e.g., to above about 10 mmol/l, the condition of hyperglycemia develops, which is one of the symptoms of diabetes mellitus. It is thus evident that maintaining the concentration of glucose in the blood at a proper level is critically important for wellness and good health.

Diabetes mellitus is a serious medical condition affecting approximately 10.5 million Americans, in which the patient is not able to maintain blood glucose levels within the normal range (normoglycemia). Approximately 10% of these patients have insulin-dependent diabetes mellitus (Type I diabetes, IDDM), and the remaining 90% have non-insulin-dependent diabetes mellitus (Type II diabetes, NIDDM). The long-term consequences of diabetes include increased risk of heart disease, blindness, end-stage renal disease, and non-healing ulcers in the extremities. The economic impact of diabetes to society has been estimated by the American Diabetes Association at approximately $45.2 billion annually (Jonsson, B., The Economic Impact of Diabetes, Diabetes Care 21(Suppl 3): C7-C10, (1998)).

Unfortunately, some individuals, either through disease, dramatic and/or sudden changes to the body (such as may be caused by injury or surgery), or for other reasons, are unable to maintain the proper level of glucose in their blood. In such instances, the amount of glucose can usually be altered, as required, in order to bring the glucose concentration to a proper level. A shot of insulin, for example, can be administered in order to decrease the glucose concentration (insulin decreases the amount of glucose in the blood). Conversely, glucose may be added directly to the blood through injection, an intravenous (IV) solution, or indirectly by eating or drinking certain foods or liquids.

Before the glucose concentration can be properly adjusted, however, an attending physician (or the patient), must know what the present glucose concentration is and whether such concentration is increasing or decreasing. The only viable techniques available for measuring glucose concentration has been by drawing a blood sample and directly measuring the amount of glucose therein, or by measuring the amount of sugar in the urine. Both measurement techniques are not only inconvenient for the patient, but also may require significant time, manpower, and the use of expensive laboratory instruments, tools or aids to complete. As a result, it is usually not possible for a physician to know immediately what the glucose concentration of a given patient is. Rather, fluid samples must first be obtained, sent to the doctor, tested or analyzed, and a report issued. Based on such report, appropriate corrective action can then be taken when needed, e.g., through insulin injections or IV supplements, to move the glucose concentration back to an acceptable level. Unfortunately, however, because of the inherent time delay involved with gathering the fluid samples, performing the analysis, and issuing the report, such corrective action may not be possible until several hours after it is first needed. Even after the report is issued, the report results may be misinterpreted, or (e.g., through transcription or analysis error) may simply be wrong. Hence, it is apparent that what is needed is a way to accurately determine the glucose concentration of a patient immediately, effectively communicate such measured concentration to a physician or other interested person (including the patient) with minimum likelihood of error, store this data to provide a medical context and provide a clear indication of a patient's health and compliance.

Even after the glucose concentration is known, the physician must still estimate how much corrective action is required until such time as a direction and rate of change of the glucose concentration level has been established. Unfortunately, to identify a trend in the glucose concentration using existing techniques, i.e., to determine whether the glucose concentration is increasing or decreasing, and at what rate, a series of the above-described body fluid measurements must first be made, and the results then analyzed. Such measuring and analyzing process only further delays any appropriate corrective action. What is clearly needed, therefore, is a glucose measurement system that provides a physician, or other medical personnel (or the patient) with a rapid measure or indication of the rate of change of the glucose concentration, and a historical context thereby immediately informing the physician whether any corrective action is needed.

Most diabetics monitor their condition by repeatedly pricking their fingers using a lancelet in order to obtain blood samples for evaluation. A major drawback to self-monitoring of glucose is that it is discontinuous and therefore meaningful historical data depends on the number of glucose measurements performed by the patient. Further, there is no way for a physician to ensure or monitor the patient to ensure it is done at the appropriate times, and the correct number of times a day.

The biggest single need in the market involves the management and use of data generated by patients performing self-BGM. As noted above, it is difficult for the doctors to wait for the reports and depend on accurate results in order to make a timely and effective decision on the treatment for the patient. A very small share of patients download their results into software programs that allow them or their HCPs to use the data in managing their diabetes. There are several reasons for this but the main culprit is inconvenience. The majority of patients are older than 55, are not very computer literate, do not understand the software or the benefits of the exercise, and finally there is no integration with health care professionals when patients are left to handle this issue by themselves.

Therefore, there is a significant need in the market for a self-BGM system that accurately determines the blood glucose in a patient, and can timely and effectively report the results to a physician in a timely manner so that the physician can make decisions in regard to the treatment required by the patient.

### SUMMARY OF THE INVENTION

In view of the several disadvantages of delays in reporting the results of self-BGM to the doctor and of keeping and maintaining this data, the present invention provides a novel device and system to quickly send the data and test results to the doctor, patient, payor or other identified party after a patient performs self-BGM.

Therefore, the present invention provides a passive wireless data management system for blood glucose meters that sends the blood glucose data using a wireless process and software through a wireless communication system. The blood glucose data is transferred to a central server which can be accessed by a doctor and/or patient, payor, other identified party or health care professional to use for monitoring blood glucose levels and adjusting the therapy for the particular patient.

Accordingly, a method of monitoring a blood glucose level of a patient is provided, the method comprising acquiring results of a test of a patient's blood glucose level at a first location, converting the results of the patient's blood glucose level to glucose data, automatically transmitting the glucose data to a second location, and providing access to the glucose data from the second location for review of the glucose data.

The method further comprises providing therapy treatment to the patient based on the review of the glucose data, and automatically transmitting the therapy treatment to the patient at the first location. The method can further comprise automatically displaying the therapy treatment to the patient at the first location. The therapy treatment can comprise a prescription or treatment techniques.

The method of monitoring a blood glucose level of a patient further comprises storing the glucose data electronically at the first location, and/or storing the glucose data electronically at the second location. The glucose data can be stored in a central server at the second location. Access can be provided to the glucose data at a location different than the first or second location.

Also provided is a blood glucose monitoring system, the system comprising a blood glucose monitoring unit for administering a blood glucose test on a patient at a first location and converting results of the test to glucose data, a transmitting unit for automatically transmitting the glucose data to a second location, and a storage unit for storing the glucose data and allowing access to the stored glucose data from the second location.

The blood glucose monitoring system further comprises an electronic device for accessing the glucose data from the second location for review of the glucose data. The blood glucose monitoring system further comprises a second transmitting unit for providing therapy treatment from the electronic device to the patient at the first location. The blood glucose monitoring system further comprises a computer processing unit at the first location for storing the glucose data. The blood glucose monitoring unit further comprises a second transmitting unit for wirelessly transmitting the glucose data to the computer processing unit for storage.

The transmitting unit can comprise a wireless communication network. The blood glucose monitoring unit can be a self blood glucose monitoring unit. The blood glucose monitoring unit can comprise an audio function that talks the patient through each step and gives the patient an audible and visual test result. The blood glucose monitoring unit constantly administers blood glucose tests on the patient at a predetermined frequency.

Also provided is a blood glucose monitoring system, the system comprising a self blood glucose monitoring unit for constantly administering blood glucose tests on a patient at predetermined frequencies at a first location and converting results of the tests to a set of glucose data, a central processing unit for storing each set of glucose data and automatically transmitting each set of glucose data, and a central server at a second location for receiving each set of glucose data from the central processing unit and storing each set of glucose data, wherein the central server allows access by a third party to each set of glucose data for review of the glucose data to provide therapy treatment for the patient based on review of the glucose data.

The above and other features of the invention, including various novel details of construction and combinations of parts, will now be more particularly described and pointed out in the claims. It will be understood that the particular device embodying the invention is shown by way of illustration only and not as a limitation of the invention. The principles and features of this invention may be employed in various and numerous embodiments without departing from the scope of the invention.

### DETAILED DESCRIPTION OF THE INVENTION

Although this invention is applicable to numerous and various types of self monitoring systems, it has been found particularly useful in the field of self-BGM systems. Therefore, without limiting the applicability of the invention to the above, the invention will be described in such environment.

Existing self-BGM techniques and devices for glucose measurements have a high level of accuracy. Many of these routine methods are accepted as standards of comparison with new devices. Management of diabetes currently relies on these methods to control the disease and minimize complications, and many of these techniques can be performed by the patient themselves. The present invention allows the patient to perform the self-BGM techniques and methods to determine their blood glucose concentration.

Advances in the field of electronics over the past several years have brought about significant changes in medical diagnostic and monitoring equipment, including arrangements for self-care monitoring of various chronic conditions. With respect to the control and monitoring of diabetes, relatively inexpensive and relatively easy-to-use blood glucose monitoring systems have become available that provide reliable information that allows a diabetic and his or her healthcare professional to establish, monitor and adjust a treatment plan (diet, exercise, and medication). More specifically, microprocessor-based blood glucose monitoring systems are being marketed which sense the glucose level of a blood sample that is applied to a reagent-impregnated region of a test strip that is inserted in the glucose monitor. When the monitoring sequence is complete, the blood glucose level is displayed by, for example, a liquid crystal display (LCD) unit. Microprocessor-based blood glucose monitoring systems are a significant advance over previously available self-care systems such as those requiring a diabetic to apply a blood sample to reagent activated portions of a test strip; wipe the blood sample from the test strip after a predetermined period of time; and, after a second predetermined period of time, determine blood glucose level by comparing the color of the reagent activated regions of the test strip with a color chart supplied by the test strip manufacturer.

Typically, currently available self-care blood glucose monitoring units include a calendar/clock circuit and a memory circuit that allows a number of blood glucose test results to be stored along with the date and time at which the monitoring occurred. The stored test results (blood glucose level and associated time and date) can be sequentially recalled for review by the blood glucose monitor user or a health professional by sequentially actuating a push button or other control provided on the monitor. In some commercially available devices, the average of the blood glucose results that are stored in the monitor (or the average of the results for a predetermined period of time) also is displayed during the recall sequence. Further, some self-care blood glucose monitors allow the user to tag the test result with an “event code” that can be used to organize the test results into categories. For example, a user might use a specific event code to identify test results obtained at particular times of the day, a different event code to identify a blood glucose reading obtained after a period of exercise, two additional event codes to identify blood glucose readings taken during hypoglycemia symptoms and hyperglycemia symptoms, etc. When event codes are provided and used, the event code typically is displayed with each recalled blood glucose test result.

Microprocessor-based blood glucose monitoring systems have advantages other than the capability of obtaining reliable blood glucose test results and storing a number of the results for later recall and review. By using low power microprocessor and memory circuits and powering the units with small, high capacity batteries (e.g., a single alkaline battery), extremely compact and light designs have been achieved that allow taking the blood glucose monitoring system to work, school, or anywhere else the user might go with people encountered by the user not becoming aware of the monitoring system. In addition, most microprocessor-based self-care blood glucose monitoring systems have a memory capacity that allows the system to be programmed by the manufacturer so that the monitor displays a sequence of instructions during any necessary calibration or system tests and during the blood glucose test sequence itself. In addition, the system monitors various system conditions during a blood glucose test (e.g., whether a test strip is properly inserted in the monitor and whether a sufficient amount of blood has been applied to the reagent impregnated portion of the strip) and if an error is detected generates an appropriate display (e.g., “retest”). A data port may be provided that allows test results stored in the memory of the microprocessor-based blood glucose monitoring system to be transferred to a data port of a personal computer or other such device for subsequent analysis.

The present invention uses a blood glucose monitor (BGM) that may include an audio function that talks the patient through each step and gives an audible and visual test result. It is preferably a microprocessor-based BGM system. The BGM system can record from 1 to millions of data points. The monitor also uses a reagent that combines the lancet and testing strip into one consumable/disposable strip. Software provided in the monitor interprets the results of the self-testing and converts the results into glucose data. The monitor may use an outlet port connection for a wire so that the glucose data can be transmitted through a wired system (USB link). The data may be sent to a computer processing unit (CPU) and can be stored in the internal memory of the CPU. The monitor might also contain an antenna or infrared transmitter or another type of electronic data sending device which could send the data to the CPU.

In the prior art, this data could be lost before being transferred to the CPU because the battery of the monitor dies, the meter is reset, or the data just gets lost. In the present invention, the data is transferred to the CPU immediately or soon after the testing.

The data on the CPU may be sent to a central server through a wired system or a wireless communication network. Alternatively, the data may be transmitted directly from the BGM to a central server. This data can be transferred immediately or soon after the patient finishes the self-testing, and can be done automatically without requiring any action from the patient. The upload can be at a predetermined frequency such as after every test, 10 times a day, or once a day. This information is then stored on a central server (such as a third party central server).

Further, other information can also be uploaded besides the glucose data, such as a patient's eating and exercise habits, what food was eaten, what exercise was performed and for how long, and any other information that a doctor, patient or other third party may want to know or inquire about.

The data on the central server can then be accessed by a doctor or healthcare professional to use for monitoring blood glucose levels, diet, exercise and adjusting therapy. This data can be accessed on a computer or database by the doctor, or any other type of electronic device, such as a palm held device, cellular phone, personal digital assistant, etc. The data can also be accessed by the patient if the patient wants to view the results as well. The doctor can then provide therapy treatments by sending prescriptions or treatment techniques back to the central server, which is sent from the central server back to the patient's CPU. This information could automatically be displayed on the patient's CPU once received by the central server and sent to the CPU.

In one embodiment, data is provided to a computer, which performs the processing of the data and displays a result on a monitor attached to the computer. The present invention is typically implemented using a computer, which generally includes one or more processors, random access memory (RAM), data storage devices (e.g., hard, floppy, and/or CD-ROM disk drives, etc.), data communications devices (e.g., modems, network interfaces, etc.), display devices (e.g., CRT, LCD display, etc.), and input devices (e.g., camera, video recorder, mouse pointing device, and keyboard). It is envisioned that attached to the computer may be other devices, such as read only memory (ROM), a video card, bus interface, printers, etc. Those skilled in the art will recognize that any combination of the above components, or any number of different components, peripherals, and other devices, may be used with the computer.

The computer operates under the control of an operating system (OS). The operating system is booted into the memory of the computer for execution when the computer is powered-on or reset. In turn, the operating system then controls the execution of one or more computer programs by the computer. The present invention is generally implemented in these computer programs, which execute under the control of the operating system and cause the computer to perform the desired functions as described above, such as the sending of the glucose data to a central server, and then automatically displaying any instructions received from the doctor or HCP. This data may also be sent and accessed through the internet when stored on a central server.

Thus, the present invention may be implemented as a method, apparatus, system, or article of manufacture using standard programming and/or engineering techniques to produce software, firmware, hardware, or any combination thereof. The term “article of manufacture” (or alternatively, “computer program product”) as used herein is intended to encompass a computer program accessible from any computer-readable device, carrier, or media, including the internet. Of course, those skilled in the art will recognize many modifications may be made to this configuration without departing from the scope of the present invention.

Those skilled in the art will recognize that the environment described above is not intended to limit the present invention. Indeed, those skilled in the art will recognize that other alternative hardware environments may be used without departing from the scope of the present invention. For example, the computer may be a portable, self-contained unit that comprises a data processing system and may be about the size of the palm of an average individual's hand.

The system described above may provide full integration and record keeping for all patients using this monitor. By registering the monitor to a patient and allowing HCP access to their test results, all future test results would be captured via automated periodic downloads to a central server that would place the data in a software application. The application would then generate reports which would be available to patient or HCP at any time, for example, during a doctor's appointment.

There would be additional benefits in the area of compliance/adherence, data quality improvement, financial management of resources for healthcare payors such as HMOs/PPos or CMS. The diabetic population has always been difficult to track due to major data gaps and this invention would almost entirely close that gap for patients using this kind of system. The system would be beneficial to patient and doctor since they can monitor treatment better. It would also be beneficial to the payor since they can monitor the use of strips and other supplies and insure none are being misused or wasted.

The above description of the present invention is only the preferred embodiment of the invention. Embodiments may include any currently or hereafter-known versions of the elements described herein. The self blood glucose monitoring systems can be invasive or non-invasive tests. The system could be a wired or wireless system, and can be accessed through a third party server or over the internet.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A method of monitoring a blood glucose level of a patient, the method comprising:
acquiring results of a test of a patient's blood glucose level at a first location;
converting the results of the patient's blood glucose level to glucose data;
automatically transmitting the glucose data to a second location; and
providing access to the glucose data from the second location for review of the glucose data.

2. The method of monitoring a blood glucose level of a patient of claim 1, further comprising:
providing therapy treatment to the patient based on the review of the glucose data.

3. The method of monitoring a blood glucose level of a patient of claim 2, further comprising:
automatically transmitting the therapy treatment to the patient at the first location.

4. The method of monitoring a blood glucose level of a patient of claim 3, further comprising:
automatically displaying the therapy treatment to the patient at the first location.

5. The method of monitoring a blood glucose level of a patient of claim 2, wherein the therapy treatment comprises a prescription.

6. The method of monitoring a blood glucose level of a patient of claim 2, wherein the therapy treatment comprises treatment techniques.

7. The method of monitoring a blood glucose level of a patient of claim 1, further comprising:
storing the glucose data electronically at the first location.

8. The method of monitoring a blood glucose level of a patient of claim 1, further comprising:
storing the glucose data electronically at the second location.

9. The method of monitoring a blood glucose level of a patient of claim 1, wherein the glucose data is stored in a central server at the second location.

10. The method of monitoring a blood glucose level of a patient of claim 1, wherein the access is provided to the glucose data at a location different than the first or second location.

11. A blood glucose monitoring system, the system comprising:
a blood glucose monitoring unit for administering a blood glucose test on a patient at a first location and converting results of the test to glucose data;
a transmitting unit for automatically transmitting the glucose data to a second location; and
a storage unit for storing the glucose data and allowing access to the stored glucose data from the second location.

12. The blood glucose monitoring system of claim 11, further comprising:
an electronic device for accessing the glucose data from the second location for review of the glucose data.

13. The blood glucose monitoring system of claim 12, further comprising:
a second transmitting unit for providing therapy treatment from the electronic device to the patient at the first location.

14. The blood glucose monitoring system of claim 11, further comprising:
a computer processing unit at the first location for storing the glucose data.

15. The blood glucose monitoring system of claim 14, wherein the blood glucose monitoring unit further comprises a second transmitting unit for wirelessly transmitting the glucose data to the computer processing unit for storage.

16. The blood glucose monitoring system of claim 11, wherein the transmitting unit comprises a wireless communication network.

17. The blood glucose monitoring system of claim 11, wherein the blood glucose monitoring unit is a self blood glucose monitoring unit.

18. The blood glucose monitoring system of claim 11, wherein the blood glucose monitoring unit comprises an audio function that talks the patient through each step and gives the patient an audible and visual test result.

19. The blood glucose monitoring system of claim 11, wherein the blood glucose monitoring unit constantly administers blood glucose tests on the patient at a predetermined frequency.

20. A blood glucose monitoring system, the system comprising:
a self blood glucose monitoring unit for constantly administering blood glucose tests on a patient at predetermined frequencies at a first location and converting results of the tests to a set of glucose data;
a central processing unit for storing each set of glucose data and automatically transmitting each set of glucose data; and
a central server at a second location for receiving each set of glucose data from the central processing unit and storing each set of glucose data;
wherein the central server allows access by a third party to each set of glucose data for review of the glucose data to provide therapy treatment for the patient based on review of the glucose data.
